(19) United States
(12) Patent Application Publication
(10) Pub. No.: US 2026/0184703 A1
(43) Pub. Date: Jul. 2, 2026

(54) PROCESS FOR THE PREPARATION OF SUBSTITUTED AMINOTHIOPHENE

(71) Applicant: ADAMA AGAN LTD., Ashdod (IL)

(72) Inventors: Michael GRABARNICK, Metar (IL); Shaoxiang WU, Haidian District, Beijing (CN); Wei LIU, Haidian District, Beijing (CN); Rui GAO, Haidian District, Beijing (CN)

(73) Assignee: ADAMA AGAN LTD., Ashdod (IL)

(21) Appl. No.: 19/119,644

(22) PCT Filed: Oct. 12, 2023
(86) PCT No.: PCT/IL2023/051071

§ 371 Date: Apr. 9, 2025

(30) Foreign Application Priority Data
Oct. 14, 2022 (WO) PCT/CN2022/125364

(51) Int. Cl. C07D 417/12

(52) U.S. Cl. CPC C07D 417/12

(57) ABSTRACT

The invention relates to a method for preparing 4-alkoxy carbonyl-3-aminothiophene of formula (I) or acid-addition salt thereof of formula (I)′, which comprises reacting the corresponding 3-oxotetrahydrothiophenes compound of formula (II) with an acid-addition salt of hydroxylamine in the presence of a polar protic acidic solvent and a dehydrating reagent.

### FIELD OF THE INVENTION

The invention relates to a process for preparing substituted 4-alkoxycarbonyl-3-aminothiophene of formula (I) or acid-addition salt thereof of formula (I)′, which are known as intermediates for active ingredients in agriculture, especially used for the preparation of herbicidal active thiencarbazone-methyl compound.

### BACKGROUND OF THE INVENTION

4-alkoxycarbonyl-3-aminothiophene are useful as intermediates in the preparation of pharmaceutical and/or agrochemical compounds.

Baker, B. R., et.al. J. Org. Chem., 18, 138-152 (1953), describes a two-step process for the preparation of 3-amino-4-methoxycarbonylthiophene hydrochloride from 3-oxo-4-methoxycarbonyltetrahydrothiophene. The procedure involves isolating 3-oximino-4-methoxycarbonyltetrahydrothiophene and reacting it with hydrogen chloride in the presence of ether and methanol. It also involves reacting 3-oxo-4-methoxycarbonyltetrahydrothiophene with hydroxylamine, which is created in situ from hydroxylamine hydrochloride and barium carbonate. The drawback of this reaction is that it is a two-step process, and both steps in the process require long reaction times.

GB 1,587,084 patent discloses the reaction of 3-oxatetrahydrothiophenes with an acid addition salt of hydroxylamine to produce 4-alkoxycarbonyl-3-aminothiophenes. The resulting oximes can then be subjected to acid treatment or naturally transformed into the corresponding amine hydrochlorides. The drawbacks of this reaction are the occurrence of decarboxylated amine as an undesirable by-product, challenging purification, and the requirement to employ a significant excess of the hydroxylamine acid addition salt.

U.S. Pat. No. 4,317,915 patent discloses the conversion of 3-oxatetrahydrothiophenes in the presence of hydroxylamine hydrohalide and an inert organic solvent to produce 4-alkoxycarbonyl-3-aminothiophenes. In addition, the reaction is carried out with an excess of the nitrogen containing base. The drawback of this reaction is the use of an excess amount of nitrogen containing base.

EP 0,298,542 patent discloses the conversion of 3-oxatetrahydrothiophenes with hydroxylamine hydrochloride and acetonitrile to produce 4-alkoxycarbonyl-3-aminothiophenes. However, the achievable yield in the process is not entirely satisfactory and it uses an excess amount of hydroxylamine hydrochloride in the reaction.

The processes described in the prior art have flaws such as long reaction times that make the process uneconomical; excessive reagent use that makes the process less efficient; use of expensive solvents hardly recovered. Thus, there is still a need for a process that obviates the shortcomings associated with the known processes.

Therefore, there is a need to develop an effective process for the preparation of 4-alkoxycarbonyl-3-aminothiophene of formula (I) or acid-addition salt thereof of formula (I)′, with higher yield and efficiency, is therefore desirable.

### SUMMARY OF THE INVENTION

The present invention provides a process for the preparation of 4-alkoxycarbonyl-3-aminothiophene of formula (I)

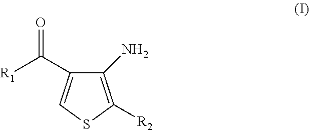

or a hydrochloride of the formula (I)′

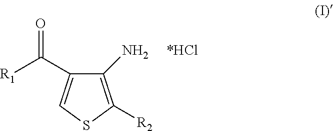

in which R₁ is C₁-C₄-alkoxy, and R₂ is C₁-C₄ alkyl;
which comprises reacting of compound of formula (II)

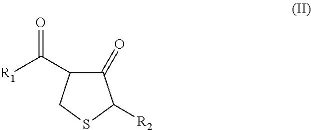

in which R₁ is C₁-C₄-alkoxy, and R₂ is C₁-C₄ alkyl;

with an acid-addition salt of hydroxylamine in the presence of a polar protic acidic solvent and a dehydrating reagent, wherein the reaction is in the absence of a base.

In an aspect, the present invention provides that R₁ and R₂ in the compound of formula (I) and formula (I)′, R₁ is C₁-C₄-alkoxy, and R₂ is selected from the group comprising methyl, ethyl, propyl, or isopropyl, preferably R₁ is methoxy, and R₂ is methyl.

In another aspect, the present invention provides that R₁ and R₂ in the compound of formula (II), R₁ is C₁-C₄-alkoxy, and R₂ is selected from the group comprising methyl, ethyl, propyl or isopropyl, preferably R₁ is methoxy, and R₂ is methyl.

In yet another aspect, the present invention provides that the acid-addition salt of hydroxylamine is selected from the group comprising hydrochloride, hydrobromide, sulfate, phosphate, or nitrate, preferably hydroxylamine hydrochloride.

In a further aspect, the present invention provides that the acid-addition salt of hydroxylamine be used in an amount of 1 to 1.5 equivalent.

In an aspect, the present invention provides that the polar protic acidic solvent is a C₁-C₄ carboxylic acid, preferably acetic acid.

In another aspect, the present invention provides that the dehydrating agent is a C₁-C₄ carboxylic acid anhydride, preferably acetic anhydride.

In yet another aspect, the present invention provides that the dehydrating reagent is used in an amount of 0.1 to 10 equivalent, preferably 0.2 to 0.5 equivalent.

In a further aspect, the present invention provides that the reaction is carried out at a temperature ranging from 60 to 100° C., preferably 80 to 85° C.

In another aspect, the present invention provides a process for the preparation of thiencarbazone-methyl comprises preparation of the compound of formula (I) and/or formula (I)′ and further converting to thiencarbazone-methyl of formula (X).

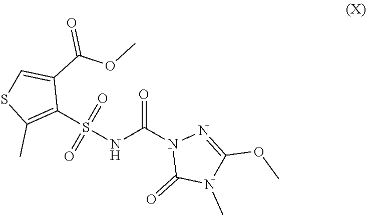 [SMILES: COC(=O)c1csc(C)c1S(=O)(=O)NC(=O)n1nc(OC)n(C)c1=O]

In a preferred aspect, the present invention provides a process for the preparation of 2-methyl-4-methoxycarbonyl-3-aminothiophene or 2-methyl-4-methoxycarbonyl-3-aminothiophene hydrochloride which comprises the reaction of 2-methyl-4-methoxycarbonyl-3-oxo tetrahydrothiophene with hydroxylamine hydrochloride in the presence of acetic acid and acetic anhydride.

### DESCRIPTION OF THE INVENTION

For the sake of clarity, specific terminology is resorted to in describing the embodiments of the invention. However, it is not intended that the invention be limited to the specific terms so selected, and it is to be understood that each specific term includes all technical equivalents that operate in a similar manner to accomplish a similar purpose.

It will be understood that the terminology used herein is for the purpose of describing embodiments only and is not intended to be limiting. As used in this specification, the singular forms “a”, “an” and “the” include plural referents unless the context clearly dictates otherwise. Thus, for example, the reference to “a compound” includes one or more of such compounds.

Unless defined otherwise, all technical and scientific terms used herein have the same meaning as commonly understood by one ordinarily skilled in the art to which the invention pertains. Although other methods and materials similar to or equivalent to those described herein can be used in the practise of the present invention, the preferred materials and methods are described herein.

As used herein, the term “or” has the meaning of both “and” and “or”. It will be further understood that the terms “comprises”, “comprising”, “includes”, “including”, or any other variation thereof, are intended to cover a non-exclusive inclusion, subject to any limitation explicitly indicated. For example, a composition or a method that comprises a list of elements is not necessarily limited to only those elements but may include other elements not expressly listed or inherent to such a composition or method.

Throughout the application, descriptions of various embodiments use the term “comprising”. However, it will be understood by one of skill in the art that in some specific instances, an embodiment can alternatively be described using the language “consisting essentially of” or “consisting of.”

Accordingly, unless indicated to the contrary, the numerical parameters set forth in the following specification and attached claims are approximations that may vary depending upon the desired properties sought to be obtained. At the very least, each numerical parameter should at least be construed considering the number of reported significant digits and by applying ordinary rounding techniques.

In addition, the endpoints of all ranges directed to the same component or property herein are inclusive of the endpoints, are independently combinable, and include all intermediate points and ranges.

The term “equivalent” refers to the quantity of a substance that reacts with an arbitrary quantity (usually one mole) of another substance in a particular chemical reaction.

As used herein, the term “alkyl” refers to straight or branched chain, saturated alkyl groups having from 1 to 4 carbon atoms, such as methyl, ethyl, propyl, isopropyl and the like.

As used herein, the term “alkoxy” refers to saturated straight or branched chain alkoxy groups having from 1 to 4 carbon atoms, such as methoxy, ethoxy, propoxy, isopropoxy, and the like. As used herein, the term “carboxylic acid” refers to saturated straight or branched chain carboxylic acid groups having from 1 to 4 carbon atoms, such as formic acid, acetic acid, and the like.

As used herein, the term “carboxylic acid anhydride” refers to saturated straight or branched chain carboxylic acid anhydride groups having from 1 to 4 carbon atoms, such as acetic anhydride, propionic anhydride, and the like.

As used herein, the term “dehydrating reagent” refers to a reagent that quench the water molecule from the reaction mass and prevents the side reaction. The dehydrating reagent is C₁-C₄ carboxylic acid anhydride, such as acetic anhydride, propionic anhydride, and the like.

4-alkoxycarbonyl-3-aminothiophene are useful as intermediates in the preparation of pharmaceutical and/or agrochemical compounds. For example, it is used as an intermediate in the preparation of herbicidal active thiencarbazone-methyl compound. U.S. Pat. No. 4,428,963 patent discloses the use of certain 3-aminothiophenes in the preparation of thiophene derivatives that are useful as blood lipid lowering agents and as antiobesity agents.

The present invention provides a process for preparing a 4-alkoxycarbonyl-3-aminothiophene of formula (I)

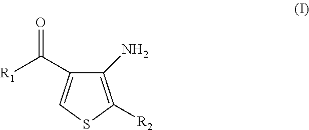

or a hydrochloride of the formula (I)′

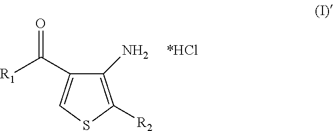

in which R₁ is C₁-C₄-alkoxy, and R₂ is C₁-C₄ alkyl;
which comprises reacting of compound of formula (II)

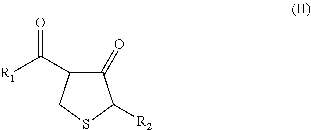

in which R₁ is C₁-C₄-alkoxy, and R₂ is C₁-C₄ alkyl;
with an acid-addition salt of hydroxylamine in the presence of a polar protic acidic solvent and a dehydrating reagent, wherein the reaction is in the absence of a base.

In an embodiment, the present invention provides that R₁ and R₂ in the compound of formula (I) and formula (I)′, R₁ is C₁-C₄-alkoxy, and R₂ is selected from the group comprising methyl, ethyl, propyl, or isopropyl, preferably R₁ is methoxy, and R₂ is methyl.

In another embodiment, the present invention provides that R₁ and R₂ in the compound of formula (II), R₁ is C₁-C₄-alkoxy, and R₂ is selected from the group comprising methyl, ethyl, propyl or isopropyl, preferably R₁ is methoxy, and R₂ is methyl.

The foregoing reaction is carried out with an acid-addition salt of hydroxylamine in the presence of a polar protic acidic solvent and a dehydrating reagent.

The acid-addition salt of hydroxylamine is selected from the group comprising hydrochloride, hydrobromide, sulfate, phosphate, or nitrate, preferably hydroxylamine hydrochloride. The polar protic acidic solvent is C₁-C₄ carboxylic acids, such as formic acid, acetic acid, and the like, preferably acetic acid. The dehydrating reagent is a C₁-C₄ carboxylic acid anhydride, such as acetic anhydride, propionic anhydride, and the like, preferably acetic anhydride.

In an embodiment, the present process provides that the acid-addition salt of hydroxylamine is used in an amount of 1 to 1.5 equivalent. In a further embodiment, the acid-addition salt of hydroxylamine is used in an amount of 1 to 1.4 equivalent. In yet another embodiment, the acid-addition salt of hydroxylamine is used in an amount of 1.1 to 1.4 equivalent. In a preferred embodiment, the acid-addition salt of hydroxylamine is used in an amount of 1.2 equivalent.

In an embodiment, the present invention makes the process more efficient and possesses the ability to reduce the excess hydroxylamine hydrochloride to zero.

In an embodiment, the present process provides that the molar ratio between the acid-addition salt of hydroxylamine and the compound of formula (I) or formula (I′) is from about 1:1 to 1.5:1. In a further embodiment, the molar ratio between the acid-addition salt of hydroxylamine and the compound of formula (I) or formula (I′) is from about 1.1:1 to 1.5:1. In yet another embodiment, the molar ratio between the acid-addition salt of hydroxylamine and the compound of formula (I) is from about 1.1:1 to 1.4:1. In a preferred embodiment, the molar ratio between the acid-addition salt of hydroxylamine and the compound of formula (I) or formula (I′) is about 1.2:1.

In another embodiment, the present process provides that the dehydrating reagent is used in an amount of 0.1 to 10 equivalent. In another embodiment, the dehydrating reagent is used in an amount of 0.2 to 8 equivalent. In a further embodiment, the dehydrating reagent is used in an amount of 0.2 to 5 equivalent. In yet another embodiment, the dehydrating reagent is used in an amount of 0.2 to 3 equivalent. In another embodiment, the dehydrating reagent is used in an amount of 0.2 to 1 equivalent. In a preferred embodiment, the dehydrating reagent is used in an amount of 0.2 to 0.5 equivalent.

In an embodiment, the reaction to form the compound of formula (I) from the compound of formula (II) is conducted at a temperature from about 60° C. to about 100° C., preferably from about 65° C. to about 95° C., more preferably from about 70° C. to about 90° C. In a preferred embodiment, the reaction is conducted at a temperature ranging from about 75° C. to about 85° C.

In a preferred embodiment, the reaction is conducted at a temperature ranging from about 80° C. to about 85° C.

The process according to the present invention is generally carried out under atmospheric pressure. However, it is also possible to carry out the process according to the invention under elevated or reduced pressure. In an embodiment, the process for preparing 4-alkoxycarbonyl-3-aminothiophene of formula (I) or acid-addition salt thereof of formula (I)′ may be conducted at a pressure from about 1 bar to about 10 bar. In another embodiment, the process may be conducted at a pressure from about 1 bar to about 5 bar. In yet another embodiment, the process for preparing 4-alkoxycarbonyl-3-aminothiophene of formula (I) or acid-addition salt thereof of formula (I)′ may be conducted at atmospheric pressure. In a further embodiment, the process may be conducted at about 10 bar. In certain embodiments, it may be preferred that the process is conducted at pressures less than atmospheric pressure. For example, the process may be carried out at 0.7 bar, 0.75 bar, 0.8 bar, 0.9 bar or 0.95 bar.

In a further embodiment, the present invention provides the resulting compound of formula (I) or formula (I′) is present at a purity of at least 60%, at least 80%, at least 85%, at least 88%, at least 89%, at least 90%, at least 91%, at least 92%, at least 93%, at least 94%, at least 95%, at least 96%, at least 97%, at least 98%, or at least 99%.

In yet another embodiment, the present invention provides the compound of formula (I) or formula (I′)′ as being used for the preparation of thiencarbazone-methyl compound.

In a preferred embodiment, the present invention provides a process for the preparation of 2-methyl-4-methoxycarbonyl-3-aminothiophene or 2-methyl-4-methoxycarbonyl-3-aminothiophene hydrochloride, which comprises the reaction of 2-methyl-4-methoxycarbonyl-3-oxo tetrahydrothiophene with hydroxylamine hydrochloride in the presence of acetic acid and acetic anhydride.

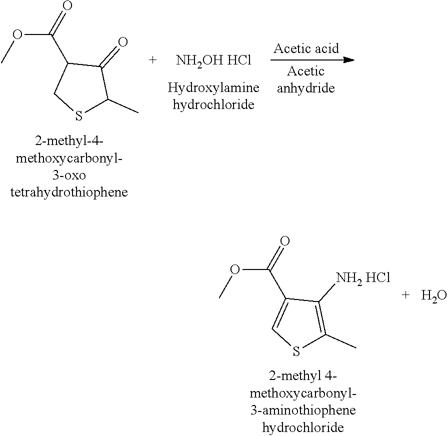

The present process is advantageous in that it is highly efficient, providing a shorter reaction time as well as fewer effluents. The resulting compound of formula (II) is subsequently reacted with the acid-addition salt of hydroxylamine in the presence of acetic acid and acetic anhydride as a dehydrating reagent that removes the water at each equilibrium step. The reduction of water concentration in the reaction mass not only moves the equilibrium but also prevents the side reactions. It also provides high conversion and selectivity with a higher yield of the end product.

One-pot process for preparing a 4-alkoxycarbonyl-3-aminothiophene of formula (I)

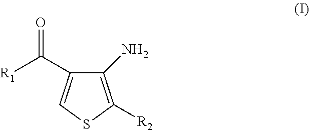

or a hydrochloride of the formula (I)

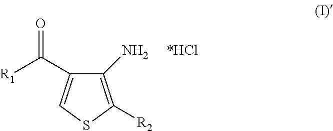

in which R₁ is C₁-C₄-alkoxy, and R₂ is C₁-C₄ alkyl;
which comprises reacting of compound of formula (II)

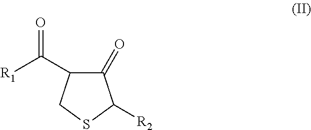

in which R₁ is C₁-C₄-alkoxy, and R₂ is C₁-C₄ alkyl;
with an acid-addition salt of hydroxylamine in the presence of a polar protic acidic solvent and a dehydrating reagent, wherein the reaction is in the absence of a base. In a preferred embodiment, R₁ is methoxy, and R₂ is methyl. In another embodiment, the acid-addition salt of hydroxylamine is hydroxylamine hydrochloride. In a further embodiment, the polar protic acidic solvent is acetic acid, and the dehydrating reagent is acetic anhydride. The present one-pot process reduces the cost of production, simplifies workup, and minimizes any effluent disposal problems.

According to an embodiment, the resultant product comprises a compound of formula (I) or formula (I′) with a purity of at least 60%, at least 80%, at least 85%, at least 88%, at least 89%, at least 90%, at least 91%, at least 92%, at least 93%, at least 94%, at least 95%, at least 96%, at least 97%, at least 98%, or at least 99%.

The progress of the reaction of synthesis of (I)/(I)′ can be monitored using any suitable method, which can include, for example, chromatographic methods such as, e.g., high performance liquid chromatography (HPLC), thin layer chromatography (TLC), and the like. In yet another embodiment, the compound of formula (I) or formula (I′) can be isolated from the reaction mixture by any conventional technique well-known in the art. Such isolation techniques can be selected, without limitation, from the group consisting of extraction, crystallization, or precipitation by concentration, cooling or antisolvent addition; filtration; centrifugation, and a combination thereof, followed by drying.

In yet another embodiment, the compound of formula (I) or formula (I′) can be optionally purified by any conventional technique well-known in the art. Such purification techniques can be selected, without limitation, from the group consisting of precipitation, crystallization, extraction, slurrying, washing in a suitable solvent, filtration through a packed-bed column, dissolution in an appropriate solvent, re-precipitation by addition of a second solvent in which the compound is insoluble, and a combination thereof.

In another embodiment, the present invention provides a process for preparation of thiencarbazone-methyl of formula (X) comprising preparation of aforementioned compound of formula (I) or formula (I)′ and further converting to thiencarbazone-methyl of formula (X). The compound of formula (I) or formula (I)′ can be converted to formula (X) such as described in the art for example in PCT Application No. WO 2001/005788.

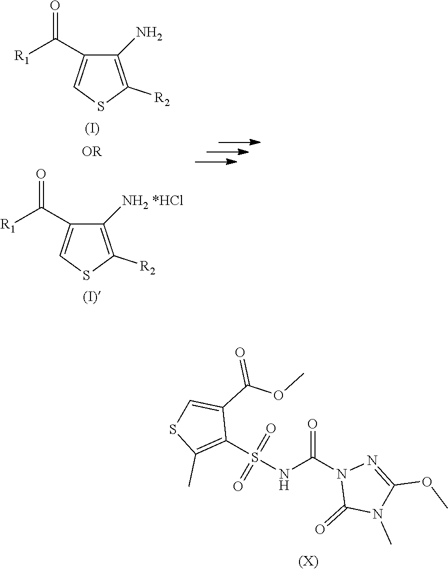

The following examples illustrate the practice of the present invention in some of its embodiments but should not be construed as limiting the scope of the present invention. From consideration of the specification and examples, other embodiments will be apparent to one skilled in the art. It is intended that the specification, including the examples, be considered exemplary only without limiting the scope and spirit of the present invention.

An exemplary experimental procedure for producing 2-methyl-4-methoxycarbonyl-3-aminothiophene hydrochloride is described as follows:

### Example 1

Acetic acid (834 g) and hydroxylamine hydrochloride (135.5 g, 1.95 mol, 1.02 eq.) were placed in a four-necked flask. The reaction mixture was heated to 80 to 85° C. The mixture of acetic anhydride (93.8 g, 0.92 mol, 0.48 eq.) and 2-methyl-4-methoxycarbonyl-3-oxo tetrahydrothiophene (333.8 g, 1.92 mol, 1 eq.) was dropped at 80 to 85° C. during 1 h. The reaction mixture was held at 80 to 85° C. for 2 h and the reaction progress was monitored by HPLC. The reaction mixture was cooled to 5 to 10° C. and held for 1.5 h. The suspension was filtered, and the product was dried in a vacuum to obtain 2-methyl-4-methoxycarbonyl-3-aminothiophene hydrochloride. The yield of 2-methyl-4-methoxycarbonyl-3-aminothiophene hydrochloride is 83%.

### Example 2

Acetic acid (980 g) and hydroxylamine hydrochloride (160.1 g, 2.30 mol, 1.2 eq.) were placed in a four-necked flask. The reaction mixture was heated to 80 to 85° C. The mixture of acetic anhydride (195.8 g, 1.92 mol, 1 eq.) and 2-methyl-4-methoxycarbonyl-3-oxo tetrahydrothiophene (333.8 g, 1.92 mol, 1 eq.) was dropped at 80 to 85° C. during 1 h. The reaction mixture was held at 80 to 85° C. for 4 h and the reaction progress was monitored by HPLC. The reaction mixture was cooled to 5 to 10° C. and held for 1.5 h. The suspension was filtered, and the product was dried in a vacuum to obtain 2-methyl-4-methoxycarbonyl-3-aminothiophene hydrochloride. The yield of 2-methyl-4-methoxycarbonyl-3-aminothiophene hydrochloride is 84%.

### Comparative Example

Acetic acid (834 g) and hydroxylamine hydrochloride (126.8 g, 1.82 mol, 0.95 eq.) were placed in a four-necked flask. The reaction mixture was heated to 80 to 85° C. The mixture of acetic anhydride (93.8 g, 092 mol, 0.48 eq.) and 2-methyl-4-methoxycarbonyl-3-oxo tetrahydrothiophene (333.8 g, 1.92 mol, 1 eq.) was dropped at 80 to 85° C. during 1 h. The reaction mixture was held at 80 to 85° C. for 3 h and the reaction progress was monitored by HPLC. The reaction mixture was cooled to 5 to 10° C. and held for 1.5 h. The suspension was filtered, and the product was dried in a vacuum to obtain 2-methyl-4-methoxycarbonyl-3-aminothiophene hydrochloride. The yield of 2-methyl-4-methoxycarbonyl-3-aminothiophene hydrochloride is 76%.

We claim:

1. A process for preparing a 4-alkoxycarbonyl-3-aminothiophene of formula (I) 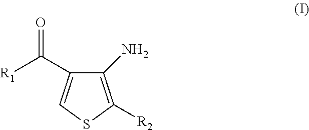
or a hydrochloride of the formula (I)′ 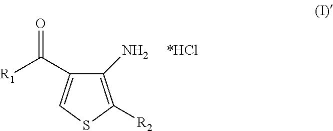
in which R₁ is C₁-C₄-alkoxy, and R₂ is C₁-C₄ alkyl;
which comprises reacting of compound of formula (II) 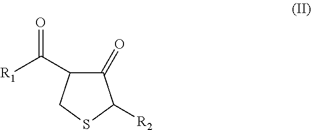
in which R₁ is C₁-C₄-alkoxy, and R₂ is C₁-C₄ alkyl;
with an acid-addition salt of hydroxylamine in the presence of a polar protic acidic solvent and a dehydrating reagent, wherein the reaction is in the absence of a base.

2. The process as claimed in claim 1, wherein the compound of formula (I) and formula (I)′, wherein
R₁ is methoxy, ethoxy, propoxy, or butoxy;
R₂ is selected from the group comprising methyl, ethyl, propyl or isopropyl.

3. The process as claimed in claim 2, wherein the compound of formula (I) and formula (I)′, wherein R₁ is methoxy, and R₂ is methyl.

4. The process as claimed in claim 1, wherein the compound of formula (II), wherein
R₁ is methoxy, ethoxy, propoxy, or butoxy;
R₂ is selected from the group comprising methyl, ethyl, propyl or isopropyl.

5. The process as claimed in claim 4, wherein the compound of formula (II), wherein R₁ is methoxy, and R₂ is methyl.

6. The process as claimed in claim 1, wherein the acid-addition salt of hydroxylamine is used in an amount of 1 to 1.5 equivalent.

7. The process as claimed in claim 1, wherein the acid-addition salt of hydroxylamine is selected from the group comprising hydrochloride, hydrobromide, sulfate, phosphate or nitrate.

8. The process as claimed in claim 1, wherein the polar protic acidic solvent is C₁-C₄ carboxylic acid.

9. The process as claimed in claim 8, wherein C₁-C₄ carboxylic acid is acetic acid.

10. The process as claimed in claim 1, wherein the dehydrating reagent is C₁-C₄ carboxylic acid anhydride.

11. The process as claimed in claim 10, wherein the C₁-C₄ carboxylic acid anhydride is acetic anhydride.

12. The process as claimed in claim 1, wherein the dehydrating reagent is used in an amount of 0.1 to 10 equivalent.

13. The process as claimed in claim 12, wherein the dehydrating reagent is used in an amount of 0.2 to 0.5 equivalent.

14. The process as claimed in claim 1, wherein the reaction is carried at a temperature of from 60 to 100° C.

15. The process as claimed in claim 14, wherein the reaction is carried at a temperature of from 80 to 85° C.

16. The process as claimed in claim 1, wherein the compound of formula (II) is reacted with hydroxylamine hydrochloride in the presence of acetic acid and acetic anhydride.

17. The process as claimed in claim 1, wherein the compound of formula (I) is further converted to thiencarbazone-methyl of formula (X).

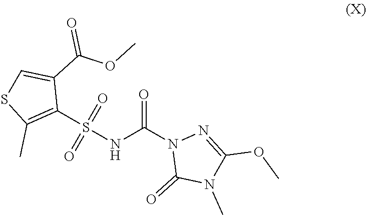 [SMILES: COC(=O)c1csc(C)c1S(=O)(=O)NC(=O)n1nc(OC)n(C)c1=O]

18. A process for preparation of compound of thiencarbazone-methyl comprising preparation of compound of formula (I) as claimed in claim 1 and further converting to thiencarbazone-methyl of formula (X). 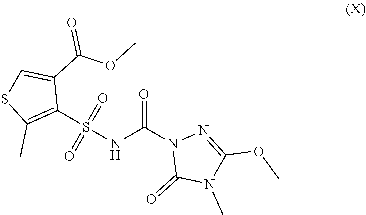 [SMILES: COC(=O)c1csc(C)c1S(=O)(=O)NC(=O)n1nc(OC)n(C)c1=O]
